(19) United States
(12) Patent Application Publication
(10) Pub. No.: US 2023/0312765 A1
(43) Pub. Date: Oct. 5, 2023

(54) COMPOUND COMPRISING AN EPOXIDE GROUP

(71) Applicant: COMPAGNIE GENERALE DES ETABLISSEMENTS MICHELIN, Clermont-Ferrand (FR)

(72) Inventors: FRANCOIS JEAN-BAPTISTE-DIT-DOMINIQUE, Clermont-Ferrand (FR); SERGEY IVANOV, Clermont-Ferrand (FR); RUSLAN NASYBULLIN, Clermont-Ferrand (FR)

(21) Appl. No.: 18/011,740

(22) PCT Filed: Jun. 24, 2021
(86) PCT No.: PCT/FR2021/051160

§ 371 Date: Dec. 20, 2022

(30) Foreign Application Priority Data
Jun. 29, 2020 (FR) FR2006790

(51) Int. Cl. C08C 19/22; C08K 3/36; C08K 3/04; C08K 3/22; C08K 5/548; C08K 5/18; C08K 5/3437; C08K 5/101; C08K 5/47; C07D 303/23

(52) U.S. Cl. CPC C08C 19/22; C08K 3/36; C08K 3/04; C08K 3/22; C08K 5/548; C08K 5/18; C08K 5/3437; C08K 5/101; C08K 5/47; C07D 303/23; C08K 2003/2296

(57) ABSTRACT

Disclosed is a compound of formula (I) below:

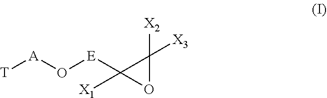

in which: T is chosen from the group consisting of CN⁺—O⁻, CH═NOH and CHO; A represents a C₆-C₁₄ arenediyl ring optionally substituted with one or more identical or different, preferably saturated, linear or branched aliphatic hydrocarbon chains; represents a divalent C₅-C₁₂ hydrocarbon group optionally comprising one or more heteroatoms; and X₁, X₂ and X₃, which may be identical or different, represent a hydrogen atom, a C₁-C₆ alkyl or a C₆-C₁₄ aryl. Also disclosed is a process for synthesizing compounds of formula (I), a modified polymer obtained by grafting the compound of formula (I) with T=CN⁺—O⁻, a composition based on the modified polymer and an additive, and to a composition based on at least one additive and at least one compound of formula (I) with T=CN⁺—O⁻.

The field of the present invention is that of modifying agents for functionalizing unsaturated polymers, i.e. polymers bearing unsaturated carbon-carbon bonds in their chains. More precisely, these modifying agents are 1,3-dipolar compounds. The invention also relates to a process for synthesizing these compounds and to the synthetic intermediates thereof.

Modifying the structure of a polymer is particularly sought for when it is desired to bring together a polymer and a filler to form a composition. This modification may make it possible to improve, for example, the dispersion of the filler in a polymer matrix, thus resulting in a more homogeneous material and ultimately improving the properties of the composition.

Modifying the structure of polymers may notably be performed by means of functionalizing agents (or modifying agents), coupling agents or star-branching agents or post-polymerization agents, notably for the purpose of obtaining good interaction between the polymer thus modified and the filler, whether it is carbon black or a reinforcing mineral filler.

Many functionalizing agents have been proposed for improving the interaction between the filler and the polymer.

For example, WO2019102132A1 discloses as functionalizing agent an aromatic nitrile oxide compound comprising an epoxide ring, the epoxide ring being connected to the aromatic ring bearing the nitrile oxide via a divalent group —OCH₂—. This functionalizing agent, 2-(glycidyloxy)-1-naphthonitrile oxide, when grafted onto a styrene-butadiene copolymer, improves the reinforcement index of a composition containing such a modified copolymer and significantly reduces the non-linearity of that composition compared to a composition comprising an unmodified styrene-butadiene copolymer. The improved reinforcement and non-linear behaviour of this composition are obtained by maintaining the hysteresis properties at a level that is virtually identical to that of a composition comprising an unmodified styrene-butadiene copolymer.

However, there is an ongoing need for modified polymers, in particular elastomers and notably modified diene elastomers, which provide compositions with improved hysteresis properties relative to the prior art compositions.

Indeed, since fuel economy and the need to protect the environment have become a priority, it is necessary to ensure a rolling resistance which is as low as possible. The rubber compositions used for the manufacture of pneumatic tyres must thus have a hysteresis that is as low as possible in order to limit fuel consumption.

Obtaining a rubber composition with a hysteresis that is as low as possible, while at the same time maintaining a good level of other properties, such as reinforcement and stiffness, is an ongoing challenge for tyre manufacturers. Indeed, it is known that a decrease in the hysteresis of rubber compositions may be accompanied by a decrease in the baked stiffness. However, a tread must be rigid enough to ensure a good level of road behaviour of the tyre.

One aim of the present invention is thus to propose novel polymer modifying agents which, when grafted onto these polymers, make it possible to obtain compositions which have an improved reinforcement index and improved hysteresis properties without reducing the baked stiffness properties.

Continuing its research, the Applicant discovered, surprisingly, that an aromatic nitrile oxide, having an epoxide ring linked to the aromatic group bearing the nitrile oxide via a specific —O-alkanediyl group of a specific length, when grafted onto an elastomer including unsaturated carbon-carbon bonds, makes it possible to obtain compositions which simultaneously have an improved reinforcement index and improved hysteresis properties. In addition, advantageously, the composition thus obtained also shows an improvement in the baked stiffness properties.

The present invention thus relates to these nitrile oxide compounds and also to the synthetic intermediates thereof. Thus, a first subject of the present invention is a compound of formula (I)

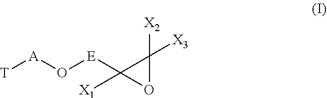

in which:
T is chosen from the group consisting of CN⁺—O⁻, CH═NOH and CHO;
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

Advantageously, a preferred compound of formula (I) is a compound of formula (Ia)

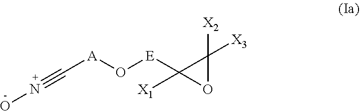

in which:
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

Advantageously, a preferred compound of formula (I) is a compound of formula (Ib)

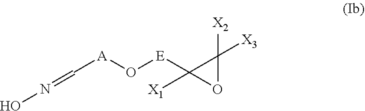

in which:
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

Advantageously, a preferred compound of formula (I) is a compound of formula (Ic)

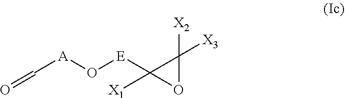

A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

Another subject of the present invention relates to a modified polymer, preferably an elastomer, notably a diene elastomer, obtained by grafting at least one compound of formula (Ia) defined previously onto at least one unsaturated carbon-carbon bond of the chain of an initial polymer.

Another subject of the present invention relates to a composition based on at least one additive and at least one polymer, preferably an elastomer, notably a diene elastomer, obtained by grafting at least one compound of formula (Ia).

Another subject of the present invention is a composition based on at least one additive and a compound of formula (Ia).

Another subject of the present invention is a process for preparing a compound of formula (Ia), said process comprising at least one reaction of a compound of formula (Ib) with an oxidizing agent in the presence of at least one organic solvent SL1 according to the reaction scheme:

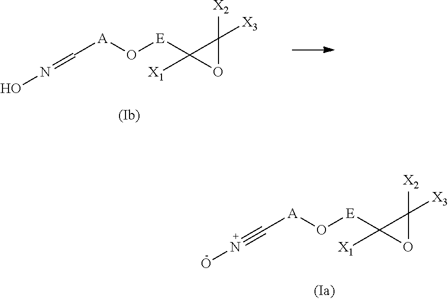

where:
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

As mentioned above, a first subject of the present invention thus relates to a compound of formula (I)

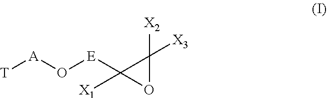

in which:
T is chosen from the group consisting of CN⁺O⁻, CH═NOH and CHO;
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

In the present text, unless expressly indicated otherwise, all the percentages (%) indicated are mass percentages (%).

Furthermore, any interval of values denoted by the expression “between a and b” represents the range of values extending from more than a to less than b (i.e. limits a and b excluded), whereas any interval of values denoted by the expression “from a to b” means the range of values extending from a up to b (i.e. including the strict limits a and b).

The compounds comprising carbon mentioned in the description may be of fossil or biobased origin. In the latter case, they may be partially or totally derived from biomass or may be obtained from renewable starting materials derived from biomass. Polymers, plasticizers, fillers, etc. are notably concerned.

The expression “composition based on” should be understood as meaning a composition 20 including the mixture and/or the product of the in situ reaction of the various constituents used, some of these constituents being able to react and/or being intended to react with each other, at least partially, during the various phases of manufacture of the composition; the composition thus possibly being in the totally or partially crosslinked state or in the non-crosslinked state.

For the purposes of the present invention, the expression “part by weight per hundred parts by weight of elastomer” (or phr) should be understood as meaning the part by mass per hundred parts by mass of elastomer.

The term “1,3-dipolar compound” is understood according to the definition given by the IUPAC. By definition, a 1,3-dipolar compound comprises a dipole. When T is CN⁺—O⁻, the dipole is a nitrile oxide.

For the purposes of the present invention, the term “hydrocarbon chain” means a chain comprising one or more carbon atoms and one or more hydrogen atoms.

The expression “Cᵢ-Cⱼ alkyl” denotes a linear, branched or cyclic hydrocarbon group comprising from i to j carbon atoms; i and j being integers.

The expression “Cᵢ-Cⱼ aryl” denotes an aromatic group including from i to j carbon atoms; i and j being integers.

The term “alkanediyl” means a hydrocarbon group derived from an alkane in which two hydrogen atoms have been removed. An alkanediyl is thus a divalent group.

The invention and the advantages thereof will be readily understood in the light of the description and the implementation examples that follow.

In the compound of formula (I), the group T is chosen from the group consisting of CN⁺—O⁻; CH═NOH and CHO, also represented in the following manner

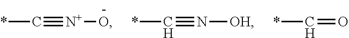

the symbol (*) representing the attachment to A.

In accordance with formula (I), the compound according to the invention comprises the group A which represents a C₆-C₁₄ arenediyl ring optionally substituted with one or more identical or different, preferably saturated, linear or branched aliphatic hydrocarbon chains.

Within the meaning of the present invention, the term “arenediyl ring” means a monocyclic or polycyclic aromatic hydrocarbon group derived from an arene in which two hydrogen atoms have been removed. An arenediyl ring is thus a divalent group.

The term “monocyclic or polycyclic aromatic hydrocarbon group” means one or more aromatic rings the backbone of which consists of carbon atoms. In other words, there are no heteroatoms in the backbone of the ring. The arenediyl ring may be monocyclic, i.e. consisting of a single ring, or polycyclic, i.e. consisting of several fused aromatic hydrocarbon rings; such fused rings then share at least two successive carbon atoms. These rings may be ortho-fused or ortho- and peri-fused. The arenediyl ring comprises from 6 to 14 carbon atoms.

The arenediyl ring may be unsubstituted, partially substituted or fully substituted. An arenediyl ring is partially substituted when one or two or more hydrogen atoms (but not all the atoms) are replaced with one or two or more, preferably saturated, linear or branched aliphatic hydrocarbon chains. Said chains are also called substituents. If all the hydrogen atoms are replaced with said chains, then the arenediyl ring is fully substituted. The substituents of the arenediyl ring may be identical to or different from each other.

Preferably, when the arenediyl ring is substituted with one or more hydrocarbon chain(s), which may be identical or different, independently of each other, this or these chain(s) are inert with respect to the epoxide ring and the chemical group represented by the symbol T (referred to for greater brevity as group T hereinbelow).

For the purposes of the present invention, “hydrocarbon chain(s) that are inert with respect to the epoxide ring and to the group T” means a hydrocarbon chain which does not react with either said epoxide ring or with said group T. Thus, said hydrocarbon chain which is inert with respect to said ring and to said group T is, for example, a hydrocarbon chain which does not bear any alkenyl or alkynyl functions that can react with said ring or said group T. Preferably, such hydrocarbon chains are aliphatic, saturated, linear or branched, and may comprise from 1 to 24 carbon atoms.

Preferably, A represents a C₆-C₁₄ arenediyl ring, optionally substituted with one or more identical or different saturated C₁-C₂₄ hydrocarbon chains. Even more preferentially, the group A is a C₆-C₁₄ arenediyl ring, optionally substituted with one or more identical or different substituents, the substituents being C₁-C₁₂, preferably C₁-C₆ and even more preferentially C₁-C₄ alkyls.

Preferably, the compound of formula (I) according to the invention is chosen from the compounds of formula (II) and the compounds of formula (III)

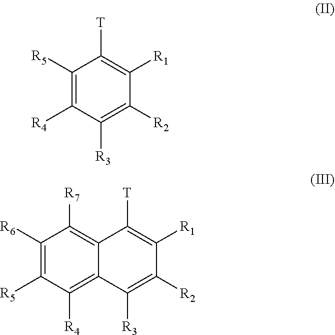

in which:
a group chosen from R₁ to R₅ of formula (II) and a group chosen from R₁ to R₇ of formula (III) denote the group of formula (IV) below:

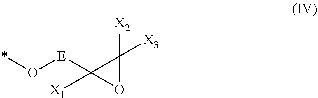

in which T, E, X₁, X₂ and X₃ are as defined previously and hereinbelow and the symbol (*) represents the attachment of the group (IV) to the rest of the molecule (II) or (III);
the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a, preferably saturated, linear or branched aliphatic hydrocarbon chain.

Preferably, in the compounds of formulae (II) and (III), the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a saturated, linear or branched C₁-C₂₄ aliphatic hydrocarbon chain.

Even more preferentially, in the compounds of formulae (II) and (III), the four groups of formula (II) chosen from R₁ to R₅ other than the one denoting the group of formula (IV) and the six groups of formula (III) chosen from R₁ to R₇ other than the one denoting the group of formula (IV), which may be identical or different, are chosen from the group consisting of a hydrogen atom, and C₁-C₁₂, preferably C₁-C₆ and even more preferentially C₁-C₄ alkyls.

Even more preferentially, in the compounds of formulae (II) and (III), the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a methyl.

According to a preferred embodiment of the invention, in formula (II), R₂ represents a group of formula (IV) and R₁, R₃, R₄ and R₅, which may be identical or different, represent a hydrogen atom or a, preferably saturated, linear or branched C₁-C₂₄ aliphatic hydrocarbon chain. More preferentially, R₂ represents a group of formula (IV) and R₁, R₃, R₄ and R₅, which may be identical or different, are chosen from the group consisting of a hydrogen atom and a C₁-C₁₂, more preferentially C₁-C₆ and even more preferentially C₁-C₄ alkyl.

Even more preferentially in this embodiment, R₂ represents a group of formula (IV), R₄ represents a hydrogen atom and R₁, R₃ and R₅ represent a, preferably saturated, linear or branched C₁-C₂₄ aliphatic hydrocarbon chain. Even more preferentially, R₂ represents a group of formula (IV), R₄ represents a hydrogen atom and R₁, R₃ and R₅, which may be identical or different, represent a C₁-C₁₂, more preferentially C₁-C₆ and even more preferentially C₁-C₄, alkyl.

According to another preferred embodiment of the invention, in formula (III), R₁ represents a group of formula (IV) and R₂ to R₇, which may be identical or different, represent a hydrogen atom or a, preferably saturated, linear or branched C₁-C₂₄ aliphatic hydrocarbon chain. More preferentially, R₁ represents a group of formula (IV) and R₂ to R₇, which may be identical or different, are chosen from the group consisting of a hydrogen atom and a C₁-C₁₂, more preferentially C₁-C₆ and even more preferentially C₁-C₄ alkyl. Even more preferentially in this embodiment, R₁ represents a group of formula (IV) and R₂ to R₇, which are identical, represent a hydrogen atom.

In the compounds of formulae (I), (II) and (III), E represents a divalent C₅-C₁₂ hydrocarbon group which may optionally contain one or more heteroatoms. For the purposes of the present invention, the term “divalent hydrocarbon group” means a spacer group (or a linking group) forming a bridge between the oxygen atom attached to A and the epoxide ring bearing the groups X₁, X₂ and X₃, this spacer group E comprising from 5 to 12 carbon atoms. This spacer group may be a C₅-C₁₂ hydrocarbon chain, which is preferably saturated, linear or branched, which may optionally contain one or more heteroatom(s), for instance N, O and S. Said hydrocarbon chain may optionally be substituted, provided that the substituents do not react with the group T and the epoxide ring as defined above.

Preferentially, in the compounds of formulae (I), (II) and (III), E represents a divalent C₅-C₁₀, preferably C₅-C₉, more preferentially C₆-C₉, even more preferentially C₇-C₉ hydrocarbon group, which may optionally contain one or more heteroatom(s), for instance N, O and S.

More preferentially, in the compounds of formulae (I), (II) and (III), E represents a C₅-C₁₀ alkanediyl, preferably a C₅-C₉ alkanediyl, more preferentially a C₆-C₉ alkanediyl, even more preferentially a C₇-C₉ alkanediyl.

Among the compounds of formula (I), particular preference is given to the compounds of formula (Ia)

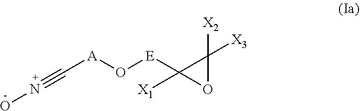

with
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

Surprisingly, in compound (Ia), when E represents a divalent group containing 5 to 12 carbon atoms, the compound of formula (Ia), when grafted onto a polymer, preferably an elastomer, notably a diene elastomer, gives the compositions based on said grafted polymer improved hysteresis and reinforcement properties compared with the compositions of the prior art. Surprisingly, the improvement in these properties is not achieved at the expense of the baked stiffness properties.

Preferentially, in formula (Ia), A represents a C₆-C₁₄ arenediyl ring, optionally substituted with one or more identical or different saturated C₁-C₂₄ hydrocarbon chains. Even more preferentially, the group A is a C₆-C₁₄ arenediyl ring, optionally substituted with one or more identical or different substituents, the substituents being C₁-C₁₂, preferably C₁-C₆ and even more preferentially C₁-C₄ alkyls.

Preferably, the compound of formula (Ia) is chosen from the compounds of formulae (IIa) and (IIIa):

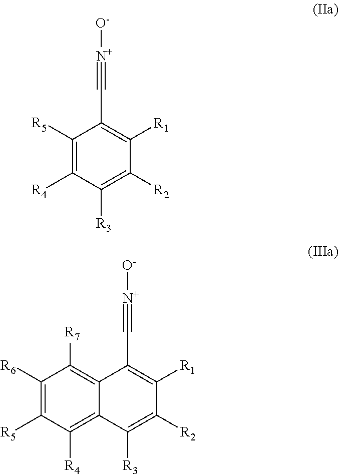

in which:
a group chosen from R₁ to R₅ of formula (IIa) and a group chosen from R₁ to R₇ of formula (IIIa) denote the group of formula (IV) below:

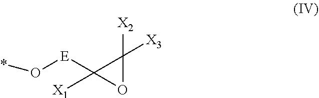

in which E, X₁, X₂ and X₃ are as defined previously and the symbol (*) represents the attachment to (IIa) or to (IIIa);
the four groups of formula (IIa) chosen from R₁ to R₅ other than the one denoting the group of formula (IV) and the six groups of formula (IIIa) chosen from R₁ to R₇ other than the one denoting the group of formula (IV), which may be identical or different, represent, independently of each other, a hydrogen atom or a, preferably saturated, linear or branched aliphatic hydrocarbon chain.

Preferably, in the compounds of formulae (IIa) and (IIIa), the four groups of formula (IIa) chosen from R₁ to R₅ other than the one denoting the group of formula (IV) and the six groups of formula (IIIa) chosen from R₁ to R₇ other than the one denoting the group of formula (IV), which may be identical or different, represent, independently of each other, a hydrogen atom or a saturated, linear or branched C₁-C₂₄ aliphatic hydrocarbon chain.

Even more preferentially, in the compounds of formulae (IIa) and (IIIa), the four groups of formula (IIa) chosen from R₁ to R₅ other than the one denoting the group of formula (IV) and the six groups of formula (IIIa) chosen from R₁ to R₇ other than the one denoting the group of formula (IV), which may be identical or different, are chosen from the group consisting of a hydrogen atom and C₁-C₁₂, preferably C₁-C₆ and even more preferentially C₁-C₄ alkyls.

Even more preferentially, in the compounds of formulae (IIa) and (IIIa), the four groups of formula (IIa) chosen from R₁ to R₅ other than the one denoting the group of formula (IV) and the six groups of formula (IIIa) chosen from R₁ to R₇ other than the one denoting the group of formula (IV), which may be identical or different, represent, independently of each other, a hydrogen atom or a methyl.

According to a preferred embodiment of the invention, in formula (IIa), R₂ represents a group of formula (IV) and R₁, R₃, R₄ and R₅, which may be identical or different, represent a hydrogen atom or a, preferably saturated, linear or branched C₁-C₂₄ aliphatic hydrocarbon chain. More preferentially, R₂ represents a group of formula (IV) and R₁, R₃, R₄ and R₅, which may be identical or different, are chosen from the group consisting of a hydrogen atom and a C₁-C₁₂, more preferentially C₁-C₆ and even more preferentially C₁-C₄ alkyl.

Even more preferentially in this embodiment, R₂ represents a group of formula (IV), R₄ represents a hydrogen atom and R₁, R₃ and R₅ represent a, preferably saturated, linear or branched C₁-C₂₄ aliphatic hydrocarbon chain. Even more preferentially, R₂ represents a group of formula (IV), R₄ represents a hydrogen atom and R₁, R₃ and R₅, which may be identical or different, represent a C₁-C₁₂, more preferentially C₁-C₆ and even more preferentially C₁-C₄, alkyl.

According to another preferred embodiment of the invention, in formula (IIIa), R₁ represents a group of formula (IV) and R₂ to R₇, which may be identical or different, represent a hydrogen atom or a, preferably saturated, linear or branched C₁-C₂₄ aliphatic hydrocarbon chain. More preferentially, R₁ represents a group of formula (IV) and R₂ to R₇, which may be identical or different, are chosen from the group consisting of a hydrogen atom and a C₁-C₁₂, more preferentially C₁-C₆ and even more preferentially C₁-C₄ alkyl. Even more preferentially in this embodiment, R₁ represents a group of formula (IV) and R₂ to R₇, which are identical, represent a hydrogen atom.

In the compounds of formulae (Ia), (IIa) and (IIIa), E represents a divalent C₅-C₁₀ hydrocarbon group which may optionally contain one or more heteroatoms. Preferentially, this spacer group may be a C₅-C₉ hydrocarbon chain, which is preferably saturated, linear or branched, which may optionally contain one or more heteroatom(s), for instance N, O and S. Said hydrocarbon chain may optionally be substituted, provided that the substituents do not react with the nitrile oxide group and the epoxide ring as defined above.

Preferably, in the compounds of formulae (Ia), (IIa) and (IIIa), E represents a divalent C₅-C₁₀, preferably C₅-C₉, more preferentially C₆-C₉ and even more preferentially C₇-C₉ hydrocarbon group, which may optionally contain one or more heteroatom(s), for instance N, O and S. Surprisingly, when E is this divalent group as described above, then the compounds of formulae (Ia), (IIa) and (IIIa), when grafted onto a polymer, preferably an elastomer, notably a diene elastomer, give the compositions based on said grafted polymer improved hysteresis and reinforcement properties relative to the compositions of the prior art. Surprisingly, the improvement in these properties is not achieved at the expense of the baked stiffness properties.

More preferentially, in the compounds of formulae (Ia), (IIa) and (IIIa), E represents a C₅-C₁₀ alkanediyl, preferably a C₅-C₉ alkanediyl, more preferentially a C₆-C₉ alkanediyl and even more preferentially a C₇-C₉ alkanediyl. Surprisingly, when E is this alkanediyl as described above, then the compounds of formulae (Ia), (IIa) and (IIIa), when grafted onto a polymer, preferably an elastomer, notably a diene elastomer, give the compositions based on said grafted elastomer improved hysteresis and reinforcement properties relative to the compositions of the prior art. Surprisingly, the improvement in these properties is not achieved at the expense of the baked stiffness properties.

Preferentially, in the compounds of formulae (I), (II) and (III) and in the preferred compounds (Ia), (IIa) and (IIIa), X₁, X₂ and X₃, which may be identical or different, are chosen from the group consisting of a hydrogen atom, C₁-C₆ alkyls and C₆-C₁₄ aryls.

Preferentially, in the compounds of formulae (I), (II) and (III) and in the preferred compounds (Ia), (IIa) and (IIIa), X₁, X₂ and X₃, which may be identical or different, are chosen from the group consisting of a hydrogen atom, methyl, ethyl and phenyl.

According to a preferred embodiment of the invention, in the compounds of formulae (I), (II) and (III) and in the preferred compounds (Ia), (IIa) and (IIIa), X₁, X₂ and X₃ are identical and represent a hydrogen atom.

According to another preferred embodiment of the invention, in the compounds of formulae (I), (II) and (III) and in the preferred compounds (Ia), (IIa) and (IIIa), X₁ and X₂ represent a hydrogen atom and X₃ represents a phenyl.

According to another embodiment of the invention, in the compounds of formulae (I), (II) and (III) and in the preferred compounds (Ia), (IIa) and (IIIa), X₃ is a hydrogen atom, and Xi and X₂, which may be identical or different, represent a hydrogen atom or a methyl.

Even more preferably, the compound of formula (I) may be the compound of formula (IIIa) in which the group R₁ is the group of formula (IV) with the group E representing C₅-C₁₀ alkanediyl, preferably a C₅-C₉ alkanediyl, more preferentially a C₆-C₉ alkanediyl, even more preferentially a C₇-C₉ alkanediyl, the groups X₁, X₂ and X₃, which may be identical or different, are chosen from the group consisting of a hydrogen atom, C₁-C₆ alkyls and C₆-C₁₄ aryls (preferably chosen from the group consisting of a hydrogen atom and C₁-C₆ alkyls), and the groups R₂ to R₇, which may be identical or different, represent a hydrogen atom.

A particularly preferred compound of formula (Ia) is the compound of formula (V):

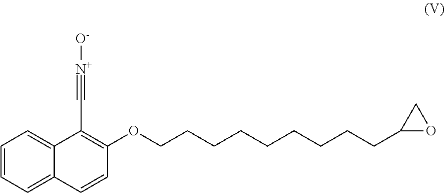 [SMILES: [O-][N+]#Cc1c(OCCCCCCCCCC2CO2)ccc2ccccc12]

Among the compounds of formula (I), the compounds of formula (Ib) below are of particular interest since they are intermediates for the synthesis of the preferred compounds of formula (Ia):

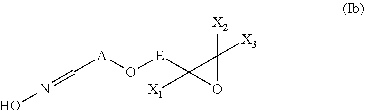

where
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

The preferred modes of A, E, X₁, X₂ and X₃ described for formulae (I), (II) and (III) also apply to the compounds of formula (Ib).

The compounds of formula (Ic) above are also of particular interest since they are intermediates for the synthesis of the preferred compounds of formula (Ia):

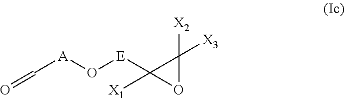

where
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

The preferred modes of A, E, X₁, X₂ and X₃ described for formulae (I), (II) and (III) also apply to the compounds of formula (Ic).

Another subject of the present invention is a process for preparing a compound of formula (Ia), said process comprising at least one reaction (d) of a compound of formula (Ib) with an oxidizing agent in the presence of at least one organic solvent SL1 according to the following reaction scheme:

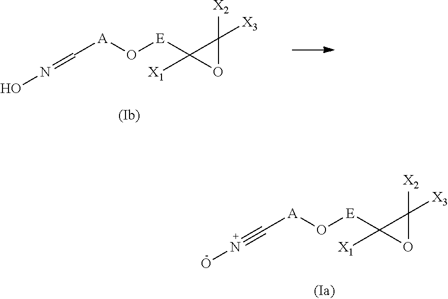

where:
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

The preferred forms of A, E, X₁, X₂ and X₃ as described above also apply to the process for preparing a compound of formula (Ia) from a compound of formula (Ib).

Preferably, said oxidizing agent is chosen from sodium hypochlorite, N-bromosuccinimide in the presence of a base, N-chlorosuccinimide in the presence of a base, and aqueous hydrogen peroxide solution in the presence of a catalyst. More preferentially, the catalyst is chosen from the group consisting of sodium hypochlorite and N-bromosuccinimide in the presence of a base.

Preferentially, the base may be triethylamine.

Advantageously, the amount of oxidizing agent is from 1 to 5 molar equivalents, preferentially from 1 to 2 molar equivalents, relative to the molar amount of the compound of formula (Ib).

Preferentially, the organic solvent SL1 is chosen from chlorinated solvents and solvents of ester, ether and alcohol type, more preferentially chosen from dichloromethane, trichloromethane, ethyl acetate, butyl acetate, diethyl ether, isopropanol and ethanol, even more preferentially chosen from ethyl acetate, trichloromethane, dichloromethane and butyl acetate.

Preferably, the compound of formula (Ib) represents from 1% to 30% by weight, preferably from 1% to 20% by weight, relative to the total weight of the combination comprising said compound of formula (Ib), said organic solvent SL1 and said oxidizing agent.

The compound of formula (Ib) may notably be obtained from a preparation process comprising at least one reaction (c) of a compound of formula (Ic) with an aqueous solution of hydroxylamine NH₂OH (compound of formula (VI)) according to the following reaction scheme:

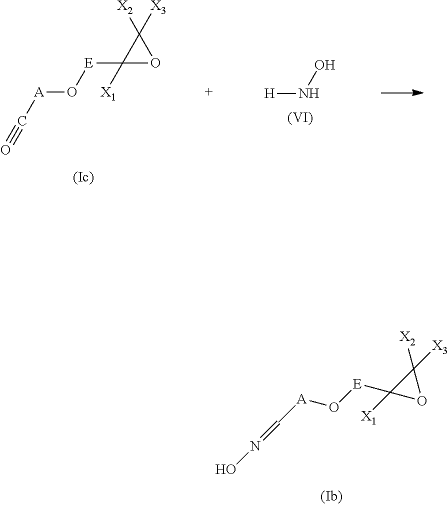

where
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

The preferred forms of A, E, X₁, X₂ and X₃ as described above also apply to the processes for preparing a compound of formula (Ib) from a compound of formula (Ic).

Preferentially, the addition of hydroxylamine (compound of formula (VI)) is performed at a temperature ranging from 1° C. to 100° C., more preferentially between 20° C. and 70° C.

The compound of formula (Ic) may be obtained by a preparation process comprising at least a reaction (b) of the compound of formula (VII) with a compound of formula (VIII) in the presence of at least one base and at a temperature ranging from 20° C. to 150° C. according to the following reaction scheme:

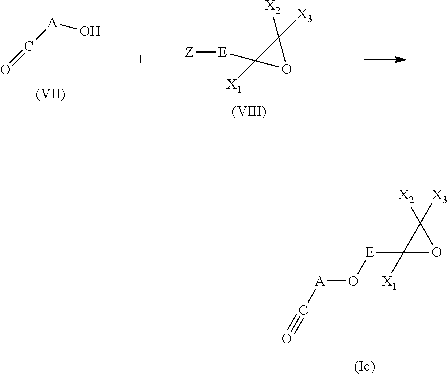

where:
A represents a C₆-C₁₄ arenediyl ring optionally substituted with one or more identical or different, preferably saturated, linear or branched aliphatic hydrocarbon chains;
E represents a divalent C₅-C₁₂ hydrocarbon group optionally comprising one or more heteroatoms;
X₁, X₂ and X₃, which may be identical or different, represent a hydrogen atom, a C₁-C₆ alkyl or a C₆-C₁₄ aryl;
Z represents a nucleofugal group.

The preferred forms of A, E, X₁, X₂ and X₃ also apply to the process for preparing a compound of formula (Ic) from the compounds of formula (VIII) and the compounds of formula (VII).

The term “nucleofugal group” means a leaving group. The group Z may be chosen from chlorine, bromine, iodine, fluorine, the mesylate group, the tosylate group, the acetate group and the trifluoromethylsulfonate group. Preferably, Z is bromine.

The reaction between the compound of formula (VIII) and that of formula (VII) is performed in the presence of at least one base and at a temperature ranging from 20° C. to 150° C.

The base may be chosen from alkali metal alkoxides, alkali metal carbonates, alkaline-earth metal carbonates, alkali metal hydroxides, alkaline-earth metal hydroxides and mixtures thereof

Preferentially, the base is chosen from sodium methoxide, potassium carbonate and sodium hydroxide, more preferentially potassium carbonate.

Preferentially, the molar amount of base is from 1.5 to 8 molar equivalents, preferably from 2 to 6 molar equivalents, relative to the molar amount of compound of formula (VII).

According to one embodiment, it is possible to add one or more catalysts chosen from a catalyst of silver salt type (such as silver oxide Ag₂O), a phase-transfer catalyst of quaternary ammonium type, and mixtures thereof.

The compounds of formula (VII) as defined above are commercially available from suppliers such as Sigma-Aldrich, Merck, Chimieliva, etc.

The compound of formula (VIII) may be obtained by epoxidation of the corresponding alkene of formula (IX) according to the reaction scheme below. The synthesis of a compound comprising an epoxide ring from its corresponding alkene is well known. For example, this epoxidation may be performed in the presence of a peracid such as meta-chloroperbenzoic acid, peracetic acid or performic acid. Another well-known technique is the use of dimethyldioxirane.

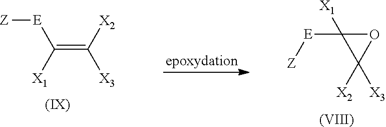

where
E represents a divalent C₅-C₁₂ hydrocarbon group optionally comprising one or more heteroatoms;
X₁, X₂ and X₃, which may be identical or different, represent a hydrogen atom, a C₁-C₆ alkyl or a C₆-C₁₄ aryl;
Z represents a nucleofugal group as described above.

The preferred forms of E, X₁, X₂ and X₃ also apply to the process for preparing a compound of formula (VIII) from the compounds of formula (IX).

The compounds of formula (IX) are commercially available from suppliers such as Sigma-Aldrich and ABCR.

As explained previously, the compounds of formula (Ia) and the preferred embodiments thereof, in particular the compound of formula (V), are used as functionalizing agent. They may be grafted onto one or more polymers comprising at least one unsaturated carbon-carbon bond; in particular, this polymer may be an elastomer and more particularly a diene elastomer. The compounds of the invention advantageously make it possible to obtain grafted polymers (also called modified polymers), in particular elastomers, notably grafted diene elastomers, irrespective of the initial microstructure of the polymer, the only condition being that the polymer comprises at least one unsaturated carbon-carbon bond, preferably a carbon-carbon double bond.

The grafting of the polymer comprising at least one unsaturated carbon-carbon bond takes place by reaction of the initial polymer with the compound of formula (Ia) and preferred embodiments thereof, in particular the compound of formula (V). The grafting of these compounds is performed by [3+2] cycloaddition of the nitrile oxide of said compounds onto an unsaturated carbon-carbon bond of the polymer chain. The mechanism of this cycloaddition is notably illustrated in WO 2012/007441. During this reaction, said compound of formula (Ia) and preferred embodiments thereof, in particular the compound of formula (V), forms covalent bonds with the polymer chain.

The grafting of the compound of formula (Ia) and preferred embodiments thereof, in particular the compound of formula (V), may be performed in bulk, for example in an internal mixer or in an external mixer, such as an open mill, or in solution. The grafting process may be performed in solution, continuously or batchwise. The modified polymer may be separated from its solution by any type of means known to those skilled in the art and in particular by a steam stripping operation.

Thus, another subject of the present invention is a modified polymer obtained by grafting at least one compound of formula (Ia) defined previously and preferred embodiments thereof, in particular the compound of formula (V), onto at least one unsaturated carbon-carbon bond of the chain of an initial polymer.

For the purposes of the present invention, the term “initial polymer chain” means the polymer chain before the grafting reaction; this chain comprises at least one carbon-carbon unsaturation that is capable of reacting with the compound of formula (Ia) described above. The initial polymer is thus the polymer serving as the starting reagent during the grafting reaction. The grafting reaction makes it possible, starting with an initial polymer, to obtain a modified polymer.

Another subject of the present invention is a composition based on an additive (preferably, the additive is a polymer, more preferentially an elastomer, notably a diene elastomer) and at least one compound of formula (Ia) defined previously (and the preferred embodiments thereof, in particular the compound of formula (V)). Another subject of the present invention is a composition based on at least one additive and at least one polymer modified with a compound of formula (Ia) defined previously (and the preferred embodiments thereof, in particular the compound of formula (V)). Preferentially, the additive may be any additive usually used in rubber compositions, notably those intended for equipping pneumatic tyres. The additives that may be used in the composition according to the invention may be plasticizers (such as plasticizing oils and/or plasticising resins), fillers (reinforcing or non-reinforcing fillers), pigments, protective agents such as antiozone waxes, chemical antiozonants, antioxidants, antifatigue agents, reinforcing resins (as described, for example, in patent application WO 02/10269), a crosslinking system, for example based on sulfur and other vulcanizing agents, and/or peroxide and/or bismaleimide.

In addition to the subjects described previously, the invention relates to at least one of the subjects described in the following embodiments:

1. Compound of formula (I) below:

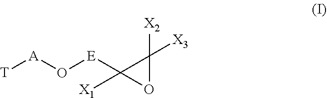

in which:
T is chosen from the group consisting of CN⁺−O⁻, CH═NOH and CHO;
A represents a C₆-C₁₄ arenediyl ring optionally substituted with one or more identical or different, preferably saturated, linear or branched aliphatic hydrocarbon chains;

E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

2. Compound of formula (I) according to embodiment 1, in which T is CH═NOH.

3. Compound of formula (I) according to embodiment 1, in which T is CHO.

4. Compound of formula (I) according to embodiment 1, in which T is CN⁺—O⁻.

5. Compound of formula (I) according to any one of embodiments 1 to 4, in which E represents a divalent C₅-C₁₀, preferably C₅-C₉, more preferentially C₆-C₉ and even more preferentially C₇-C₉ hydrocarbon group.

6. Compound of formula (I) according to any one of embodiments 1 to 4, in which E represents a C₅-C₁₀ alkanediyl, preferably a C₅-C₉ alkanediyl, more preferentially a C₆-C₉ alkanediyl and even more preferentially a C₇-C₉ alkanediyl.

7. Compound of formula (I) according to any one of the preceding embodiments, in which X₁, X₂ and X₃, which may be identical or different, are chosen from the group consisting of a hydrogen atom, C₁-C₆ alkyls and C₆-C₁₄ aryls.

8. Compound of formula (I) according to any one of embodiments 1 to 6, in which X₁, X₂ and X₃, which may be identical or different, represent a hydrogen atom, a methyl, an ethyl or a phenyl.

9. Compound of formula (I) according to any one of embodiments 1 to 6, in which X₁, X₂ and X₃ represent a hydrogen atom.

10. Compound of formula (I) according to any one of embodiments 1 to 6, in which Xi and X₂ represent a hydrogen atom and X₃ represents a phenyl.

11. Compound of formula (I) according to any one of embodiments 1 to 6, in which X₃ is a hydrogen atom and the groups Xi and X₂, which may be identical or different, represent a hydrogen atom or a methyl.

12. Compound of formula (I) according to any one of the preceding embodiments, said compound of formula (I) being chosen from the compounds of formulae (II) and (III):

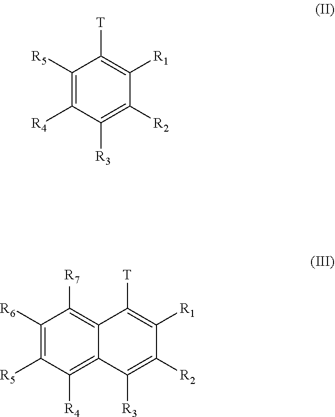

in which:
a group chosen from R₁ to R₅ of formula (II) and a group chosen from R₁ to R₇ of formula (III) denote the group of formula (IV) below:

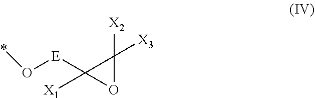

in which T, E, X₁, X₂ and X₃ are as defined in any one of embodiments 1 to 11 and the symbol (*) represents the attachment to (II) or to (III),
the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a, preferably saturated, linear or branched aliphatic hydrocarbon chain.

13. Compound of formula (I) according to embodiment 12, which compound of formula (I) is chosen from the compounds of formulae (II) and (III) in which
a group chosen from R₁ to R₅ of formula (II) and a group chosen from R₁ to R₇ of formula (III) denote the group of formula (IV) below:

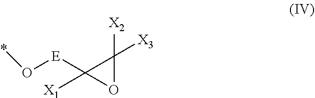

in which E, X₁, X₂ and X₃ are as defined in any one of embodiments 1 to 11 and the symbol (*) represents the attachment to (II) or to (III),
the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a methyl.

14. Compound of formula (I) according to any one of embodiments 4 to 7, in which the compound of formula (I) is the compound of formula (V).

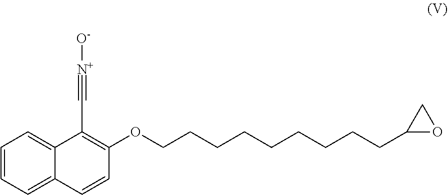 [SMILES: [O-][N+]#Cc1c(OCCCCCCCCCC2CO2)ccc2ccccc12]

15. Modified polymer obtained by grafting of at least one compound of formula (I) defined according to any one of embodiments 4 to 14 onto at least one unsaturated carbon-carbon bond of the chains of an initial polymer.

16. Modified polymer according to embodiment 15, which initial polymer is an elastomer, preferably a diene elastomer.

17. Composition based on at least one additive and on a compound of formula (I) defined according to any one of embodiments 4 to 14.

18. Composition according to embodiment 17, in which the additive is a polymer, preferably an elastomer, notably a diene elastomer.

19. Composition based on at least one additive and on a polymer as defined according to either of embodiments 15 and 16.

20. Process for preparing a compound of formula (Ia), said process comprising at least one reaction of a compound of formula (Ib) with an oxidizing agent in the presence of at least one organic solvent SL1 according to the reaction scheme:

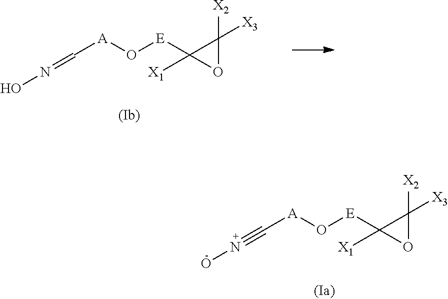

where:
A represents a C₆-C₁₄ arenediyl ring optionally substituted with one or more identical or different, preferably saturated, linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

21. Process according to embodiment 20, also comprising a step of reacting a compound of formula (Ic) with an aqueous solution of hydroxylamine NH₂OH according to the following reaction scheme:

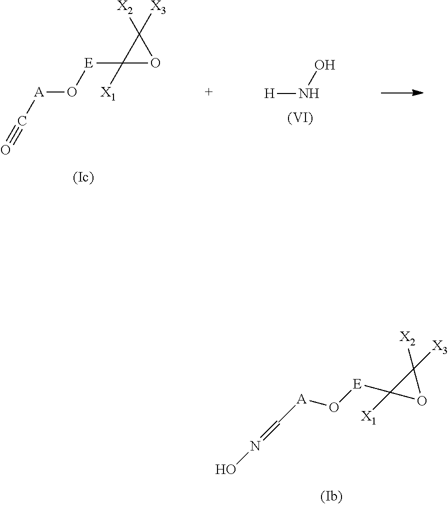

where
A represents a C₆-C₁₄ arenediyl ring optionally substituted with one or more identical or different, preferably saturated, linear or branched aliphatic hydrocarbon chains;
E represents a divalent C₅-C₁₂ hydrocarbon group optionally comprising one or more heteroatoms;
X₁, X₂ and X₃, which may be identical or different, represent a hydrogen atom, a C₁-C₆ alkyl or a C₆-C₁₄ aryl.

22. Process according to embodiment 21, also comprising a step of reacting the compound of formula (VII) with a compound of formula (VIII) in the presence of at least one base and at a temperature ranging from 20° C. to 150° C. according to the following reaction scheme:

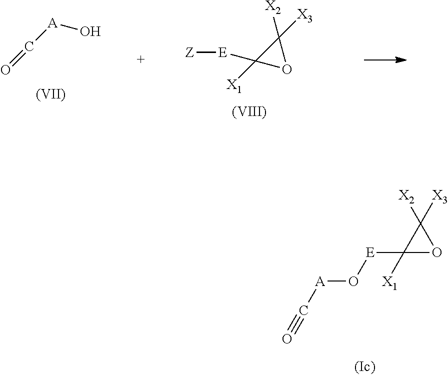

where:
A represents a C₆-C₁₄ arenediyl ring optionally substituted with one or more identical or different, preferably saturated, linear or branched aliphatic hydrocarbon chains;
E represents a divalent C₅-C₁₂ hydrocarbon group optionally comprising one or more heteroatoms;
X₁, X₂ and X₃, which may be identical or different, represent a hydrogen atom, a C₁-C₆ alkyl or a C₆-C₁₄ aryl;
Z represents a nucleofugal group.

23. Process according to any one of embodiments 20 to 22, in which E represents a divalent C₅-C₁₀, preferably C₅-C₉, more preferentially C₆-C₉ and even more preferentially C₇-C₉ hydrocarbon group.

24. Process according to any one of embodiments 20 to 22, in which E represents a C₅-C₁₀ alkanediyl, preferably a C₅-C₉ alkanediyl, more preferentially a C₆-C₉ alkanediyl and even more preferentially a C₇-C₉ alkanediyl.

25. Process according to any one of embodiments 20 to 22, in which X₁, X₂ and X₃, which may be identical or different, are chosen from the group consisting of a hydrogen atom and C₁-C₆ alkyls and C₆-C₁₄ aryls.

26. Process according to any one of embodiments 20 to 24, in which X₁, X₂ and X₃, which may be identical or different, represent a hydrogen atom, a methyl, an ethyl or a phenyl.

27. Process according to any one of embodiments 20 to 24, in which X₁, X₂ and X₃ each represent a hydrogen atom.

28. Process according to any one of embodiments 20 to 24, in which X₁ and X₂ represent a hydrogen atom and X₃ represents a phenyl.

29. Process according to any one of embodiments 20 to 24, in which X₃ is a hydrogen atom and the groups X₁ and X₂, which may be identical or different, represent a hydrogen atom or a methyl.

### EXAMPLES

The examples that follow make it possible to illustrate the invention; however, said invention shall not be limited to these examples alone.

### 1. Methods

### 1.1 Measurement of the Number-Average (Mn) and Weight-Average (Mw) Molar Masses and of the Polydispersity Index of the Elastomers

Size exclusion chromatography (SEC) is used. SEC makes it possible to separate macromolecules in solution according to their size through columns filled with a porous gel. The macromolecules are separated according to their hydrodynamic volume, the bulkiest being eluted first.

Without being an absolute method, SEC makes it possible to comprehend the distribution of the molar masses of an elastomer. The various number-average molar masses (Mn) and weight-average molar masses (Mw) may be determined from commercial standards and the polydispersity index (PDI=Mw/Mn) may be calculated via a “Moore” calibration.

### Preparation of the Elastomer Test Sample

There is no specific treatment of the elastomer sample before analysis. The latter is simply dissolved, at a concentration of approximately 1 g/l, in chloroform or in the following mixture: tetrahydrofuran+1 vol % of diisopropylamine+1 vol % of triethylamine+1 vol % of distilled water (vol %=% by volume). The solution is then filtered through a filter with a porosity of 0.45 μm before injection.

### SEC Analysis

The apparatus used is a Waters Alliance chromatograph. The elution solvent is the following mixture: tetrahydrofuran+1 vol % of diisopropylamine+1 vol % of triethylamine or chloroform, according to the solvent used for the dissolution of the elastomer. The flow rate is 0.7 ml/min, the temperature of the system is 35° C. and the analysis time is 90 min. A set of four Waters columns in series, having the commercial names Styragel HMW7, Styragel HMW6E and two Styragel HT6E, is used.

The volume of the solution of the elastomer sample injected is 100 μl. The detector is a Waters 2410 differential refractometer with a wavelength of 810 nm. The software for processing the chromatographic data is the Waters Empower system. The calculated average molar masses are relative to a calibration curve produced from PSS Ready Cal-Kit commercial polystyrene standards.

### 1.2. Characterizations of the Molecules

The structural analysis and also the determination of the molar purities of the synthesis molecules are performed by an NMR analysis. The spectra are acquired on a Brüker Avance 3 400 MHz spectrometer equipped with a “5 mm BBFO Z-grad broad band” probe. The quantitative ¹H NMR experiment uses a 30° single pulse sequence and a repetition time of 3 seconds between each of the 64 acquisitions. The samples are dissolved in a deuterated solvent, deuterated dimethyl sulfoxide (DMSO) unless otherwise indicated. The deuterated solvent is also used for the “lock” signal. For example, calibration is performed on the signal of the protons of the deuterated DMSO at 2.44 ppm relative to a TMS reference at 0 ppm. The ¹H NMR spectrum coupled with the 2D ¹H/¹³C HSQC and ¹H/¹³C HMBC experiments enable the structural determination of the molecules (cf. assignment tables). The molar quantifications are performed from the quantitative 1D ¹H NMR spectrum.

The analysis by mass spectrometry is performed by a direct-injection electrospray ionization method (DI/ESI). The analyses were performed on a Bruker HCT spectrometer (flow rate 600 μl/min, pressure of the nebulizer gas 10 psi, flow rate of the nebulizer gas 4 l/min).

### 1.3. Characterizations of the Compounds Grafted to the Diene Elastomers

The determination of the molar content of the compounds grafted to the diene elastomers is performed by an NMR analysis. The spectra are acquired on a Bruker 500 MHz spectrometer equipped with a “5 mm BBFO Z-grad CryoProbe” probe. The quantitative ¹H NMR experiment uses a simple 30° pulse sequence and a repetition time of 5 seconds between each acquisition. The samples are dissolved in deuterated chloroform (CDC₁₃) for the purpose of obtaining a “lock” signal. 2D NMR experiments made it possible to confirm the nature of the grafted unit by means of the chemical shifts of the carbon atoms and protons.

### 1.4. Dynamic Properties of the Rubber Compositions:

The dynamic properties G* and tan(δ)ₘₐₓ are measured on a viscosity analyser (Metravib VA4000) according to Standard ASTM D5992-96. The response of a sample of vulcanized composition (cylindrical test specimen with a thickness of 4 mm and with a cross section of 400 mm²), subjected to a simple alternating sinusoidal shear stress, at a frequency of 10 Hz, at a temperature of 60° C., is recorded. A strain amplitude sweep is performed from 0.1% to 100% peak-to-peak (forward cycle) and then from 100% to 0.1% peak-to-peak (reverse cycle).

The results used are the complex dynamic shear modulus G* at 25% strain (G*_{25% return}), the dynamic loss factor tan(δ) at 60° C. and the difference in modulus (ΔG*) between the values at 0.1% and 100% strain (Payne effect). For the return, the value of the complex dynamic shear modulus G* at 25% strain, denoted G*_{25% return at 60° C.} and the maximum value of the dynamic loss factor tan(δ) observed, denoted tan(δ)_{max at 60° C.}, are recorded.

The results are shown in base 100, the arbitrary value 100 being assigned to the control in order to calculate and compare subsequently tan(δ)_{max at 60° C.}, G*_{25% return at 60° C.} and ΔG* of the different samples tested.

For tan(δ)_{max at 60° C.}, the value in base 100 for the test sample is calculated according to the operation: (tan(δ)_{max at 60° C.} value of the test sample/tan(δ)_{max at 60° C.} value of the control)×100. In this way, a result of less than 100 indicates a decrease in the hysteresis, which corresponds to an improvement in the rolling resistance performance.

For G*_{25% return at 60° C.}, the value in base 100 for the test sample is calculated according to the operation: (G*_{25% return at 60° C.} value of the test sample/G*_{25% return at 60° C.} value of the control)×100. In this way, a result of greater than 100 indicates an improvement in the complex dynamic shear modulus G*_{25% return at 60° C.}, which corroborates an improvement in the stiffness of the material.

For (ΔG*), the value in base 100 for the test sample is calculated according to the operation: (ΔG* value of the test sample/ΔG* value of the control)×100. In this way, a result of less than 100 indicates a decrease in the difference in modulus, i.e. an increase in the linearization of the rubber composition.

### 1.6. Tensile Test

These tensile tests make it possible to determine the elasticity stresses. Unless otherwise indicated, they are performed in accordance with the French standard NF T46-002 of September 1988. Processing the tensile test recordings also makes it possible to plot the curve of modulus as a function of the elongation. At first elongation, the nominal secant modulus, calculated with respect to the initial cross section of the test specimen, (or apparent stress, in MPa) is measured at 100% elongation, denoted MSA100, and at 300% elongation, denoted MSA300. All these tensile test measurements are performed under the standard temperature conditions (23±2° C.) according to Standard NF T 46-002.

The MSA300/MSA100 ratio is the reinforcement index. The value in base 100 for the test sample is calculated according to the operation: (MSA300/MSA100 value of the test sample/MSA300/MSA100 value of the control)×100. In this way, a result of greater than 100 indicates an improvement in the reinforcement index.

### 2. Synthesis of the Compounds

### 2.1. Synthesis of Compound A: 2-((9-Oxiran-2-Yl)Nonyl)Oxy)-1-Naphthonitrile Oxide

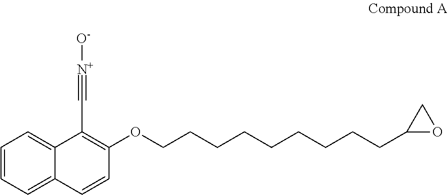 [SMILES: [O-][N+]#Cc1c(OCCCCCCCCCC2CO2)ccc2ccccc12]

Compound A is synthesized according to the following reaction scheme:

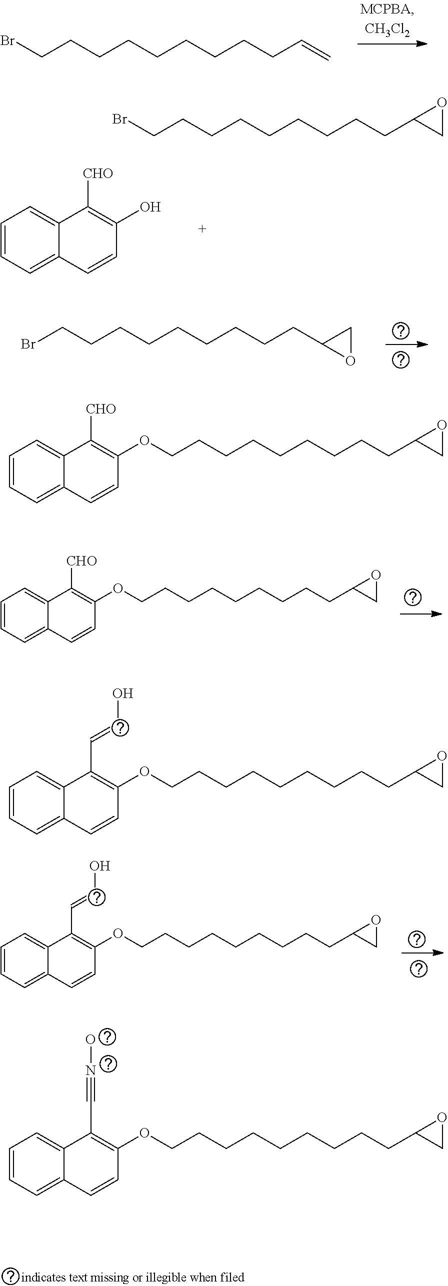

11-Bromo-1-undecene, 3-chloroperbenzoic acid, 2-hydroxy-1-naphthaldehyde, hydroxylamine and trimethylamine are commercial products. They may be obtained from Sigma-Aldrich.

### 2.1.1. Step a1: Preparation of 2-(9-Bromononyl)Oxirane

10 g of 11-bromo-1-undecene (42.9 mmol) are dissolved in 200 ml of CH₂Cl₂. 11.84 g of 3-chloroperbenzoic acid (68.6 mmol or 1.4 eq, MCPBA) are then added in several portions over about 15 minutes and the reaction medium is stirred for 15 hours. The white precipitate is filtered off and washed with CH₂Cl₂ (2×20 ml). The filtrate is then stirred in the presence of aqueous NaHSO₃ solution (40 g of NaHSO₃ in 400 ml of distilled water) for 5 hours at room temperature (20° C.). The organic phase is recovered by decantation and is left to react with a solution of NaHCO₃ (40 g) in water (400 ml) for 5 hours. After separation by decantation, the residual aqueous phase is extracted with CH₂Cl₂. The organic phases are combined, then dried with Na₂SO₄ and concentrated under reduced pressure (12 mbar; bath temperature=30° C.). A yellow oil is obtained (10.613 g, 42.90 mmol, 99% yield). The molar purity is >90% (¹H NMR).

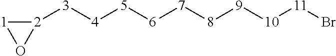

TABLE 1

| No. | δ ¹H (ppm) | δ ¹³C (ppm) |
|---|---|---|
| 1 | 2.39 and 2.68 | 47.0 |
| 2 | 2.84 | 52.3 |
| 3 | 1.49 | 32.5 |
| 4 | 1.38 | 25.9 |
| 5 | 1.25 | 28.7 to 29.4 |
| 6 | | |
| 7 | | |
| 8 | | |
| 9 | 1.36 | 28.1 |
| 10 | 1.79 | 32.8 |
| 11 | 3.34 | 33.9 |

Solvent CDCl₃

### 2.1.2 Step a2: Synthesis of 2-((9-(Oxiran-2-Yl)Nonyl)Oxy)-1-Naphthaldehyde

A suspension of 2-hydroxy-l-naphthaldehyde (11.05 g; 64.2 mmol), 2-(9-bromononyl)oxirane (15.99 g; 64.2 mmol) and K₂CO₃ (8.87 g; 64.2 mmol) in 20 ml of N,N-dimethylformamide (DMF) is heated at 70° C. for 3 hours with stirring at 500 rpm (rpm =rotations per minute). The reaction medium is then poured into 250 ml of distilled water and then extracted with ethyl acetate (4×60 ml). The organic phases were combined and then evaporated under reduced pressure (bath temperature=40° C., 10 mbar) to obtain a brown oil (24.95 g). The product is then purified by column chromatography on silica gel, eluting with a 3/1 (v/v) mixture of petroleum ether/ethyl acetate.

The residual yellow oil is triturated with petroleum ether, allowing crystallization. The precipitate is filtered off and air-dried. A yellowish solid (14.545 g, 42.7 mmol, 67% yield) is obtained. The molar purity is greater than 97 mol %.

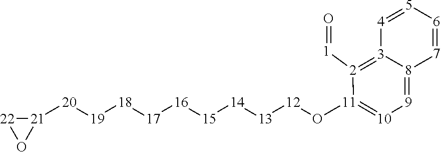

TABLE 2

| No. | δ ¹H (ppm) | δ ¹³C (ppm) |
|---|---|---|
| 1 | 10.87 | 192.1 |
| 2 | — | 116.9 |
| 3 | — | 131.6 |
| 4 | 9.22 | 125.0 |
| 5 | 7.55 | 129.8 |
| 6 | 7.35 | 124.7 |
| 7 | 7.70 | 128.2 |
| 8 | — | 128.5 |
| 9 | 7.97 | 137.4 |
| 10 | 7.21 | 113.7 |
| 11 | — | 163.7 |
| 12 | 4.17 | 69.7 |
| 13 | 1.82 | 29.4 |
| 14 | 1.46 | 25.9 |
| 15 | from 1.24 to 1.35 | 29.4 |
| 16 | | |
| 17 | | |
| 18 | | |
| 19 | 1.40 | |
| 20 | 1.46 | 32.5 |
| 21 | 2.84 | 52.3 |
| 22 | 2.40 and 2.68 | 47.1 |

Solvent: CDCl₃

### 2.1.3 Step a3: Synthesis of 2-((9-(Oxiran-2-Yl)Nonyl)Oxy)-1-Naphthaldehyde Oxime

1.521 g of hydroxylamine (23.02 mmol, i.e. 1.5 equivalents) are added, at room temperature 20° C., to a suspension of 2-((9-(oxiran-2-yl)nonyl)oxy)-1-naphthaldehyde (5.225 g, 15.35 mmol) in ethanol (50 ml). The reaction mixture is stirred for 3 hours; at the end of the reaction, a yellow solid precipitates. 30 ml of distilled water are then added and the medium is stirred for a further 10 minutes. The precipitate obtained is filtered off, washed with 2×5 ml of a distilled water/ethanol (1:1, v/v) mixture and then air-dried. A yellow solid (4.979 g, 14.01 mol, 91% yield) is obtained. The molar purity is 93% (NMR).

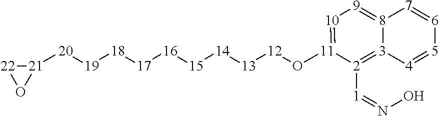

TABLE 3

| No. | δ ¹H (ppm) | δ ¹³C (ppm) |
|---|---|---|
| 1 | 8.84 | 148.1 |
| 2 | — | 113.9 |
| 3 | — | 131.7 |
| 4 | 8.80 | 125.8 |
| 5 | 7.47 | 127.8 |
| 6 | 7.32 | 124.0 |
| 7 | 7.72 | 128.3 |
| 8 | — | 129.1 |
| 9 | 7.79 | 132.0 |
| 10 | 7.19 | 114.0 |
| 11 | — | 156.6 |
| 12 | 4.08 | 69.8 |
| 13 | 1.79 | 29.4 |
| 14 | 1.47 | 26.0 |
| 15 | 1.28 | 29.2 to 29.4 |
| 16 | | |
| 17 | | |
| 18 | | |
| 19 | 1.38 | 26.0 |
| 20 | 1.47 | 32.4 |
| 21 | 2.86 | 52.4 |
| 22 | 2.42 and 2.69 | 47.1 |

Solvent: CDCl₃

### 2.1.4 Step a4: Synthesis of 2-((9-(Oxiran-2-Yl)Nonyl)Oxy)-1-Naphthonitrile N-Oxide (Compound

1.824 g of trimethylamine (18.03 mmol) and 2.037 g of N-chlorosuccinimide (15.25 mmol) are added in several portions over 10 to 12 minutes to a solution of 2-((9-(oxiran-2-yl)nonyl)oxy)-1-naphthaldehyde oxime (4.929 g; 13.87 mmol) in 100 ml CHCl₃ cooled to a temperature of 0-2° C. The reaction mixture is stirred cold for 90 minutes. The organic phase is then washed with distilled water (3×50 ml), dried with Na₂SO₄ and concentrated under reduced pressure (bath temperature=25° C.; up to 2 mbar) to give 5.005 g of a yellow solid. The product is redissolved in a minimum volume of ethyl acetate to obtain a homogeneous solution; petroleum ether is then poured in until the first signs of cloudiness appear (not very clear either, it would be preferable to indicate the volume to be poured in). This solution is filtered on a column of silica gel (1=5 cm) while eluting with an ethyl acetate/petroleum ether (1:3, v/v) mixture. The permeate is evaporated under reduced pressure (bath temperature=25° C., down to 1 mbar). A yellow solid (4.593 g, 12.99 mol, 94% yield) with a melting point of 52-53° C. is obtained. The molar purity is 92% (¹H NMR).

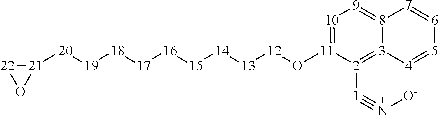

TABLE 4

| No. | δ ¹H (ppm) | δ ¹³C (ppm) |
|---|---|---|
| 1 | — | — |
| 2 | — | 96.9 |
| 3 | — | 134.3 |
| 4 | 7.93 | 124.0 |
| 5 | 7.55 | 128.4 to 128.7 |
| 6 | 7.37 | 124.9 |
| 7 | 7.76 | 128.4 to 128.7 |
| 8 | — | |
| 9 | 7.86 | 132.6 |
| 10 | 7.19 | 113.4 |
| 11 | — | 161.0 |
| 12 | 4.16 | 69.7 |
| 13 | 1.83 | 29.4 |
| 14 | 1.48 | 25.9 |
| 15 | 1.30 | 29.2 to 29.4 |
| 16 | | |
| 17 | | |
| 18 | | |
| 19 | 1.41 | 25.9 |
| 20 | 1.48 | 32.5 |
| 21 | 2.85 | 52.3 |
| 22 | 2.41 and 2.69 | 47.1 |

Solvent: CDCl₃

### 2.2. Synthesis of 2-(Glycidyloxy)-1-Naphthonitrile Oxide

2-(Glycidyloxy)-1-naphthonitrile oxide, compound B, is synthesized according to the 5 procedure described in patent application US 2012/0046418 A1.

### 3. Production of the Modified Diene Elastomers

3.1 Rubber Modified with Compound A

2-((9-(Oxiran-2-yl)nonyl)oxy)-1-naphthonitrile oxide (811 mg; 2.3 mmol, i.e. a mole fraction of 0.3 mol %), compound A obtained according to the process described above, with an NMR purity of 96 mol %, is incorporated into 50 g of natural rubber on an open mill (external mixer at 30° C.). The mixture is homogenized in 15 turnover passes. This mixing phase is followed by a heat treatment at 120° C. for 10 minutes under a press at a pressure of 10 bar.

Analysis by ¹H NMR made it possible to demonstrate a molar degree of grafting of 0.16 mol % with a molar grafting yield of 54%.

3.2 Rubber Modified with Compound B

2-(Glycidyloxy)-1-naphthonitrile oxide (564 mg, 2.34 mmol, i.e. a mole fraction of 0.3 mol %), with an NMR purity of 94 mol %, compound B, is incorporated into 50 g of natural rubber on an open mill (external mixer at 30° C.). The mixture is homogenized in 15 turnover passes. This mixing phase is followed by a heat treatment (10 minutes at 120° C.) under a press at a pressure of 10 bar.

Analysis by ¹H NMR made it possible to determine the molar degree of grafting of 0.16 mol % and the molar grafting yield of 54%.

3.3 Synthetic Polyisoprene Modified with Compound A

2-((9-(Oxiran-2-yl)nonyl)oxy)-1-naphthonitrile oxide (811 mg; 2.3 mmol, i.e. a mole fraction of 0.3 mol %), compound A obtained according to the process described above, with an NMR purity of 96 mol %, is incorporated into 50 g of synthetic polyisoprene (containing 99.35% by weight of cis 1,4-isoprene units and 0.65% by weight of 3,4-isoprene units; Mn=375 000 g/mol and PDI=3.6 measured according to the method described above) on an open mill (external mixer at 30° C.). The mixture is homogenized in 15 turnover passes. This mixing phase is followed by a heat treatment at 120° C. for 10 minutes under a press at a pressure of 10 bar.

Analysis by ¹H NMR made it possible to demonstrate a molar degree of grafting of 0.22 mol % with a molar grafting yield of 74%.

3.4 Synthetic Polyisoprene Modified with Compound B

2-(Glycidyloxy)-1-naphthonitrile oxide (564 mg; 2.34 mmol, i.e. a mole fraction of 0.3 mol %), with an NMR purity of 94 mol %, compound B, is incorporated into 50 g of synthetic polyisoprene (containing 99.35% by weight of cis 1,4-isoprene units and 0.65% by weight of 3,4-isoprene units; Mn=375 000 g/mol and PDI=3.6 measured according to the method described above) on an open mill (external mixer at 30° C.). The mixture is homogenized in 15 turnover passes. This mixing phase is followed by a heat treatment (10 minutes at 120° C.) under a press at a pressure of 10 bar.

Analysis by ¹H NMR made it possible to demonstrate a molar degree of grafting of 0.16 mol % with a molar grafting yield of 54%.

### 4. Ingredients Used in the Rubber Compositions

(1) Silica, Zeosil 1165MP, sold by Solvay;

(2) Bis[3-(triethoxysilyl)propyl] tetrasulfide (TESPT) silane, sold by Evonik under the reference Si69;

(3) Carbon black of N234 grade, sold by Cabot Corporation;

(4) N-(1,3-Dimethylbutyl)-N-phenyl-para-phenylenediamine, sold by Flexsys under the reference Santoflex 6-PPD;

(5) 2,2,4-Trimethyl-1,2-dihydroquinoline, sold by the company Flexsys;

(6) Zinc oxide (industrial grade), sold by the company Umicore;

(7) Stearin, Pristerene 4031, sold by the company Uniqema;

(8) N-Cyclohexyl-2-benzothiazolesulfenamide, sold by Flexsys under the reference Santocure CBS.

(9) natural rubber modified with compound B, obtained according to the process described in paragraph 3.1;

(10) natural rubber modified with compound A, obtained according to the process described in paragraph 3.2;

(11) synthetic polyisoprene modified with compound B, obtained according to the process described in paragraph 3.3;

(12) synthetic polyisoprene modified with compound A, obtained according to the process described in paragraph 3.4.

### 5. Test 1

The aim of this test is to show the improvement in the reinforcement of a rubber composition comprising natural rubber modified with the compound of the invention (composition C2) relative to a comparative composition (composition C1).

The contents of the various constituents of these compositions, expressed in phr, parts by weight per hundred parts by weight of elastomer, are presented in Table 5.

TABLE 5

| | C1 | C2 |
|---|---|---|
| Diene elastomer modified with compound B (9) | 100 | — |
| Diene elastomer modified with compound A (10) | — | 100 |
| Reinforcing filler (1) | 55 | 55 |
| Coupling agent (2) | 5.5 | 5.5 |
| Carbon black (3) | 3 | 3 |
| Antioxidant (4) | 1.5 | 1.5 |
| TMQ (5) | 1 | 1 |
| Paraffin | 1 | 1 |
| ZnO (6) | 2.7 | 2.7 |
| Stearic acid (7) | 2.5 | 2.5 |
| CBS (8) | 1.8 | 1.8 |
| Sulfur | 1.5 | 1.5 |

Compositions C1 and C2 comprise the same number of moles of grafted compound A or B, namely 0.3 mol %.

Compositions C1 and C2 are prepared in the following manner: the natural rubber modified with compound A or with compound B is introduced into an 85 cm³ Polylab internal mixer, filled to 70%, the initial vessel temperature of which is approximately 110° C.

Next, for each of the compositions, the reinforcing filler(s), the agent for coupling the filler with the diene elastomer and then, after kneading for one to two minutes, the various other ingredients, with the exception of the vulcanization system, are introduced. Thermomechanical work (non-productive phase) is then performed in one step, lasting a total of about 5 to 6 minutes, until a maximum drop temperature of 160° C. is reached.

The mixture thus obtained is recovered and cooled and the vulcanization system (sulfur and the sulfenamide-type accelerator) is then added on an external mixer (homofinisher) at 23° C., the whole being mixed (productive phase) for approximately 5 to 12 minutes.

The compositions thus obtained are subsequently calendered either in the form of slabs (thickness of 2 to 3 mm) or of thin sheets of rubber for measurement of their physical or mechanical properties.

The rubber properties of these compositions are measured after baking at 150° C. for 60 minutes. The results obtained are given in Table 6.

TABLE 6

| Compositions | C1 | C2 |
|---|---|---|
| MA300/MA100 | 100 | 108 |
| ΔG* | 100 | 88 |
| Tan(δ)_{max at 60° C.} | 100 | 88 |
| G*_{25% return at 60° C.} | 100 | 104 |

The rubber composition C2 of the invention shows, relative to the comparative composition C1, a significant improvement in the hysteresis properties while also showing an increase in the linearization (ΔG*) and an improvement in the reinforcement index (MA300/M100). Surprisingly, this significant improvement in hysteresis is not achieved at the expense of the 25 baked stiffness properties. On the contrary, the baked stiffness properties are even improved relative to the comparative composition.

### 5. Test 2

The aim of this test is to show the improvement in the reinforcement of a rubber composition comprising a synthetic polyisoprene modified with the compound of the invention (composition C4) relative to a comparative composition (composition C3).

The contents of the various constituents of these compositions, expressed in phr, part by weight per hundred parts by weight of elastomer, are presented in Table 7.

TABLE 7

| | C3 | C4 |
|---|---|---|
| Diene elastomer modified with compound B (11) | 100 | — |
| Diene elastomer modified with compound A (12) | — | 100 |
| Reinforcing filler (1) | 55 | 55 |
| Coupling agent (2) | 5.5 | 5.5 |
| Carbon black (3) | 3 | 3 |
| Antioxidant (4) | 1.5 | 1.5 |
| TMQ (5) | 1 | 1 |
| Paraffin | 1 | 1 |
| ZnO (6) | 2.7 | 2.7 |
| Stearic acid (7) | 2.5 | 2.5 |
| CBS (8) | 1.8 | 1.8 |
| Sulfur | 1.5 | 1.5 |

Compositions C3 and C4 comprise the same number of moles of grafted compound A or B, namely 0.3 mol %.

Compositions C3 and C4 are prepared according to the process described above for compositions C1 and C2.

The rubber properties of these compositions are measured after curing at 150° C. for 60 minutes. The results obtained are given in Table 8.

TABLE 8

| Compositions | C3 | C4 |
|---|---|---|
| MA300/MA100 | 100 | 110 |
| ΔG* | 100 | 83 |
| Tan(δ)_{max at 60° C.} | 100 | 82 |
| G*_{25% return at 60° C.} | 100 | 105 |

The rubber composition C4 of the invention shows, relative to the comparative composition C3, a significant improvement in the hysteresis properties while also showing an increase in the linearization (ΔG*) and an improvement in the reinforcement index (MA300/M100). Surprisingly, this significant improvement in hysteresis is not achieved at the expense of the baked stiffness properties. On the contrary, the baked stiffness properties are even improved relative to the comparative composition.

1.-15. (canceled)

16. A compound of formula (I) 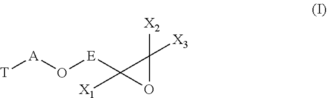
in which:
T is selected from the group consisting of CN⁺—O⁻, CH═NOH and CHO;
A represents a C₆-C₁₄ arenediyl ring optionally substituted with one or more identical or different linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

17. The compound of formula (I) according to claim 16, wherein T is CH═NOH.

18. The compound of formula (I) according to claim 16, wherein T is CHO.

19. The compound of formula (I) according to claim 16, wherein T is CN⁺—O⁻.

20. The compound of formula (I) according to claim 16, wherein E represents a divalent C₅-C₁₀ hydrocarbon group.

21. The compound of formula (I) according to claim 16, wherein E represents a C₅-C₁₀ alkanediyl.

22. The compound of formula (I) according to claim 16, wherein X₁, X₂ and X₃, which may be identical or different, are chosen from the group consisting of a hydrogen atom, a methyl, an ethyl or a phenyl.

23. The compound of formula (I) according to claim 16, wherein X₁, X₂ and X₃ represent a hydrogen atom.

24. The compound of formula (I) according to claim 16, wherein the compound of formula (I) is a compound of formulae (II) or (III) 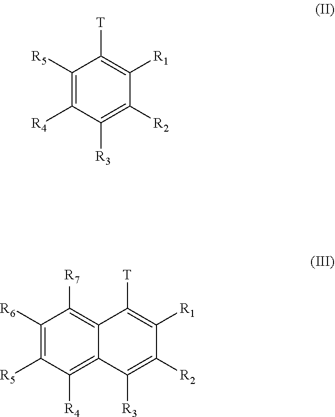
in which a group chosen from R₁ to R₅ of formula (II) and a group chosen from R₁ to R₇ of formula (III) denote the group of formula (IV) below 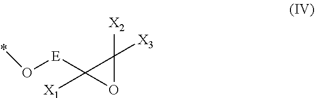
in which symbol (*) represents an attachment to (II) or to (III),
wherein the four groups of formula (II) chosen from R₁ to R₅ other than the one denoting the group of formula (IV) and the six groups of formula (III) chosen from R₁ to R₇ other than the one denoting the group of formula (IV), which may be identical or different, represent, independently of each other, a hydrogen atom or a linear or branched aliphatic hydrocarbon chain.

25. The compound of formula (I) according to claim 24, wherein the compound of formula (I) is a compound of formula (V)

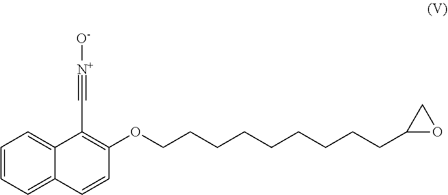 [SMILES: [O-][N+]#Cc1c(OCCCCCCCCCC2CO2)ccc2ccccc12]

26. A modified polymer obtained by grafting at least one compound of formula (I) according to claim 16 onto at least one unsaturated carbon-carbon bond of a chain of an initial polymer.

27. A composition based on at least one additive and on a compound of formula (I) according to claim 16.

28. A composition based on at least one additive and on the modified polymer according to claim 26.

29. A process for preparing a compound of formula (Ia), the process comprising at least one reaction of a compound of formula (Ib) with an oxidizing agent in the presence of at least one organic solvent SL1 according to the reaction scheme 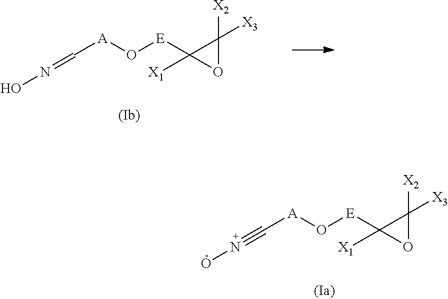
where:
A represents a C₆-C₁₄ arenediyl ring optionally substituted with one or more identical or different linear or branched aliphatic hydrocarbon chains;
E represents a C₅-C₁₂ divalent hydrocarbon group optionally comprising one or more heteroatoms; and
X₁, X₂ and X₃, which may be identical or different, represent a hydrogen atom, a C₁-C₆ alkyl or a C₆-C₁₄ aryl.

30. The process according to claim 29 further comprising a step of reacting a compound of formula (Ic) with an aqueous solution of hydroxylamine NH₂OH according to the reaction scheme

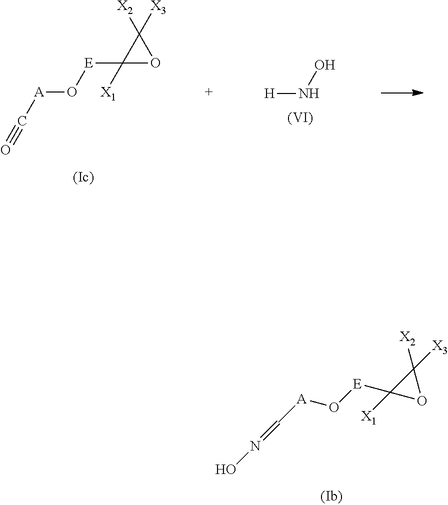

31. The process according to claim 30 further comprising a step of reacting a compound of formula (VII) with a compound of formula (VIII) in the presence of at least one base and at a temperature ranging from 20° C. to 150° C. according to the reaction scheme 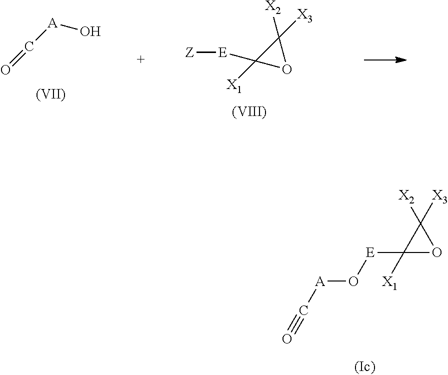
where Z represents a nucleofugal group.
